(19) United States
(12) Patent Application Publication
(10) Pub. No.: US 2007/0281016 A1
(43) Pub. Date: Dec. 6, 2007

(54) Sustained release oxycodone composition with acrylic polymer and surfactant

(75) Inventors: Huai-Hung Kao, Syosset, NY (US); Sou-Chan Chang, Babylon, NY (US); Yadi Zeng, Fort Lee, NJ (US); Fai Jim, Franklin Square, NY (US)

Correspondence Address: GUY DONATIELLO;ENDO PHARMACEUTICALS, 100 Endo Boulevard, CHADDS FORD, PA, 19317, US

(73) Assignee: Endo Pharmaceuticals Inc., a Delaware corporation, Chadds Ford, PA (US)

(21) Appl. No.: 11/447,560

(22) Filed: Jun. 6, 2006

(51) Int. Cl. A61K 31/485; A61K 9/22

(52) U.S. Cl. 424/468; 514/282

(57) ABSTRACT

The invention is a controlled release composition comprising a therapeutic amount of an active ingredient in a controlled release matrix. The matrix comprises a combination of a pharmaceutically acceptable acrylic polymer and a metal hydroxide. The amount of surfactant, relative to a given amount of acrylic polymer, is selected for and corresponds to a predetermined release rate for said active ingredient. The compound is preferably used to provide controlled release dosage of oxycodone through a matrix of ammonio methacrylic polymer and sodium lauryl sulfate.

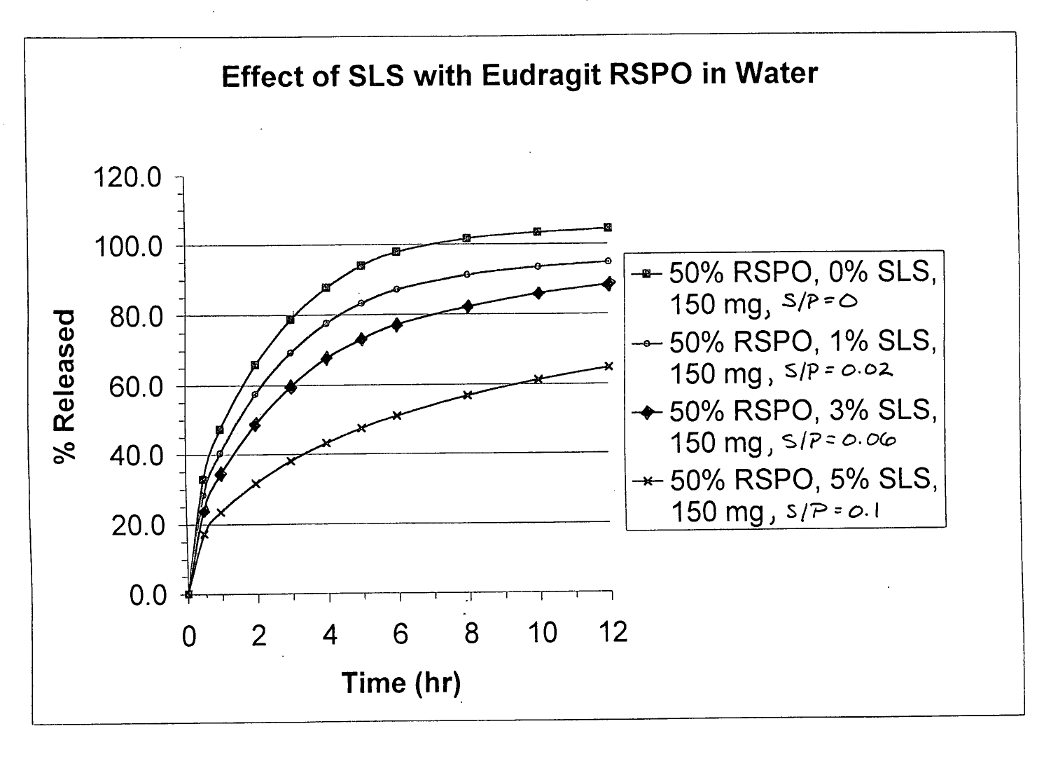

### BACKGROUND OF THE INVENTION

1. Field of the Invention

The present invention relates to a controlled release therapeutic compound employing a controlled release matrix including acrylic polymer and a surfactant. More particularly, the invention relates to a compound wherein the rate of release of an active ingredient is determined by the ratio of surfactant to acrylic polymer in the compound. Most particularly, the invention relates to a controlled release compound incorporating a therapeutic agent into a controlled release matrix including ammonio methacrylic polymer and sodium lauryl sulfate.

2. Description of the Related Art

Many medical conditions are best treated by administration of a pharmaceutical in such a way as to sustain its action over an extended period of time. Many delivery systems have been developed over time for providing such treatments. These products have become known as sustained release, controlled release, time release, etc. Each of these designations is nearly synonymous with the others. The term controlled release is used herein for convenience, and is not intended to be distinguished from the other terms in the art. Regardless of the term, the concept behind each as used herein is prolonged delivery of active ingredient over time via an oral dosage form.

Controlled release preparations provide a longer duration of pharmacological response after administration than is ordinarily experienced after the administration of an immediate release dosage form. Such extended periods of response provide for many inherent therapeutic benefits that are not achieved with short acting, immediate release products. This kind of pharmaceutical administration can be useful for treating chronic pain, such as that associated with rheumatic or arthritic conditions. Controlled-release dosage forms can also be used beneficially in the administration of a variety of drugs whose sustained action is important to their efficacy in treating many conditions.

Many physiological factors influence both the gastrointestinal transit time and the release of a drug from a controlled release dosage form, and thus influence the uptake of the drug of the patient's system. Ideally, such controlled-release dosage forms should release the active pharmaceutical ingredient at a controlled rate such that the amount of active pharmaceutical ingredient which is available in the body to treat the condition is maintained at a relatively constant and desired level over an extended period of time. That is, it is desirable that the active pharmaceutical ingredient be released at a reproducible, predictable rate.

Many controlled release formulations are known in the art. Included among these are specially coated beads or pellets, coated tablets, and ion exchange resins, wherein the slow release of the active drug is brought about through selective breakdown of, or permeation through, the coating of the preparation or through formulation with a special matrix to effect the release of the drug.

Several controlled release products incorporate fast-release and slow-release components. The combination allows for quick entry of active ingredient during initial treatment periods, while permitting a sustaining effect through later release during subsequent hours. Multiple, alternative layers of coatings and medicine, as well as coated and uncoated medicaments have been used to create this effect.

Some controlled release products use specially designed excipient matrices, which determine the rate of release. Special cellulose-derived matrices have been developed for this purpose. Specifically, plasticized ethylcellulose materials have been found to be effective. The specific composition and structure of these materials exhibit desired properties for the controlled release of the desired therapeutic agent. Other types of controlled release mechanisms are also known.

In the case of controlled release coatings, often a polymeric material, such as acrylic polymer, is used to coat a tablet or other dosage form. The particular polymer used has a disintegration of dissolution factor associated with it, correlating to the controlled rate of release of the therapeutic agent. Another benefit of these coatings is their ability to mask undesirable medicinal tastes. In some cases, the acrylic polymer has been blended into the excipient material as filler material in addition to use as the controlled release coating.

However, heretofore there has been no teaching of a controlled release formulation providing a pharmacologically active ingredient in a novel excipient matrix combining suitable proportions of an acrylic polymer and surfactant for controlling release rates of such as active ingredient.

### SUMMARY OF THE INVENTION

A controlled release composition consists of a therapeutic amount of an active ingredient in a controlled release matrix. The matrix comprises a combination of a pharmaceutically acceptable acrylic polymer and a surfactant. The amount of surfactant, relative to the amount of acrylic polymer, is selected for and corresponds to a pre-determined release rate for said active ingredient. The compound is preferably used to provide controlled release dosages of oxycodone through a matrix of ammonio methacrylic polymer and sodium lauryl sulfate.

### BRIEF DESCRIPTION OF THE DRAWING

The sole FIGURE is a graph depicting the release rate over time with various surfactant levels.

### DETAILED DESCRIPTION

The invention uses a controlled release matrix to control the release of a therapeutic ingredient. The compound can be formed into suitable solid oral dosage forms by any suitable method as is commonly known in the art. Tablets are the preferred dosage form. To obtain controlled release effects, the matrix comprises a combination of an acrylic polymer and surfactant. Reliance on a controlled release coating is unnecessary.

Many conditions may benefit from the prolonged treatment effects of controlled release products. Accordingly, many therapeutically active ingredients may be used in a controlled release manner. Pain medications are perhaps most visibly effective when administered through controlled release methods. Thus, although oxycodone and its pharmaceutically active salts are preferred, many other active ingredients may be used. Morphine and its pharmaceutically acceptable salts, oxymorphone, hydromorphone, levorphanol, codeine, hydrocodone, oxycodone, nalorphine, naloxone, naltrexone, buprenorphine, butorphanol, nalbuphine, and other common narcotics and analgesics are non-limiting examples of such active ingredients.

Unlike prior art of sustained or controlled release products, the acrylic polymer in the present invention is used as a dry excipient, and not a controlled release coating. It is to be understood that the polymer may be present as coating, but is not necessary to achieve the desired results. The acrylic polymer is combined with the surfactant into a homogeneous matrix into which the active ingredient is introduced. Surprisingly, the inventors have discovered that the rate of release of the active ingredient can be unexpectedly controlled by varying the ratio of surfactant to acrylic polymer, rather than rely solely on the rate of disintegration or dissolution of the acrylic polymer. With this discovery, the amount of active ingredient and acrylic polymer may be kept constant while achieving various release rates solely through manipulation of the amount of surfactant in the controlled release matrix. A ratio of surfactant to acrylic polymer (S/P) of approximately 0.005-0.5 by weight is contemplated by the inventors. A preferred ratio of approximately 0.02-0.2 by weight has been effective.

The preferred acrylic polymer is methacrylate based. Most specifically, an ammonio methacrylate polymer readily available under the tradename Eudragit RSPO is preferred. As discussed above, Eudragit is cited in the prior art for coatings. The polymer may account for a wide range of proportions in the tablet as long as the proper S/P ratio is mentioned.

Any surfactants may be used. Preferably, anionic surfactant is used. Most preferably, the surfactant is sodium lauryl sulfate. Minimal amounts of sodium lauryl sulfate, about 1-5% by total tablet weight, have been found to be effective. The amount of surfactant for a given amount of polymer is preferably selected based upon the desired release rate. The FIGURE illustrates the effects of compounds prepared with 0, 1, 3, and 5% sodium lauryl sulfate, corresponding to 0.02, 0.06, and 0.1 S/P respectively. As shown, the rate of release of the active ingredient is greatly reduced as the S/P ratio increases. Controlled dosages over 12, 18, and 24 hours or other increments are possible through manipulation of the S/P ratio.

Ultimately, the compound is shaped into a solid, oral dosage form according to known techniques. Dry granulation techniques are currently preferred, although the invention is not limited to these techniques alone. Other material including, but not limited to, binders, fillers, and gelling agents may be used in the matrix to form appropriately sized and shaped dosage forms. A matrix including only the acrylic polymer and the surfactant is capable of satisfactory dosage formation without the use of these additional materials, but most applications will use at least some amount of filler material. It should be appreciated that these materials are inert and generally are present mainly to aid in solid dosage (i.e. tablet) formation or other functions.

A graph plotting the percent of release versus time illustrates the effect of varying sodium lauryl sulfate concentration in the compound. All tests were performed according to USP apparatus II at a speed of 50 rpm in 900 mL dissolution medium. Four test samples were prepared at 150 mg each. Each contained approximately 10 mg oxycodone hydrochloride as the active ingredient and Eudragit RSPO as the acrylic polymer at 50% by weight of the composition. The first sample is a control without surfactant. The remaining samples had 1, 3, and 5% sodium lauryl sulfate (SLS), by weight of the composition, as the surfactant, corresponding to surfactant/polymer ratios (S/P) of 0.02, 0.06, and 0.1, respectively. The tables below show the various compositions, including various additives which are commonly added as fillers, preservatives, etc.

TABLE 1
Oxycodone Hydrochloride Extended Release Tablets,
10 mg, (control) (0 S/P)

| | Quantity | Quantity |
|---|---|---|
| Excipients | mg/tablet | (%) |
| Oxycodone Hydrochloride | 10 | 6.7% |
| Microcrystalline Cellulose | 48.5 | 32.3% |
| Ammonio Methacrylate Copolymer | 75 | 50% |
| Collodial Silicon Dioxide | 3 | 2% |
| Sodium Lauryl Sulfate | 0 | 0% |
| Povidone | 7.5 | 5% |
| Stearic Acid | 3 | 2% |
| Magnesium Stearate | 3 | 2% |
| Tablet Weight | 150 | 100% |

TABLE 2
Oxycodone Hydrochloride Extended Release Tablets,
10 mg, (0.02 S/P)

| | Quantity | Quantity |
|---|---|---|
| Excipients | mg/tablet | (%) |
| Oxycodone Hydrochloride | 10 | 6.7% |
| Microcrystalline Cellulose | 47 | 31.3% |
| Ammonio Methacrylate Copolymer | 75 | 50% |
| Collodial Silicon Dioxide | 3 | 2% |
| Sodium Lauryl Sulfate | 1.5 | 1% |
| Povidone | 7.5 | 5% |
| Stearic Acid | 3 | 2% |
| Magnesium Stearate | 3 | 2% |
| Tablet Weight | 150 | 100% |

TABLE 3
Oxycodone Hydrochloride Extended Release Tablets,
10 mg, H/P = .06

| | Quantity | Quantity |
|---|---|---|
| Excipients | mg/tablet | (%) |
| Oxycodone Hydrochloride | 10 | 6.667% |
| Microcrystalline Cellulose | 44 | 29.3% |
| Ammonio Methacrylate Copolymer | 75 | 50% |
| Collodial Silicon Dioxide | 3 | 2% |
| Sodium Lauryl Sulfate | 4.5 | 3% |
| Povidone | 7.5 | 5% |
| Stearic Acid | 3 | 2% |
| Magnesium Stearate | 3 | 2% |
| Tablet Weight | 150 | 100% |

TABLE 4
Oxycodone Hydrochloride Extended Release Tablets,
10 mg, (0.1 S/P)

| | Quantity | Quantity |
|---|---|---|
| Excipients | mg/tablet | (%) |
| Oxycodone Hydrochloride | 10 | 6.7% |
| Microcrystalline Cellulose | 41 | 27.3% |
| Ammonio Methacrylate Copolymer | 75 | 50% |
| Collodial Silicon Dioxide | 3 | 2% |
| Sodium Lauryl Sulfate | 7.5 | 5% |
| Povidone | 7.5 | 5% |
| Stearic Acid | 3 | 2% |
| Magnesium Stearate | 3 | 2% |
| Tablet Weight | 150 | 100% |

From the above, it is readily apparent that during the tests only the amount of surfactant, sodium lauryl sulfate was varied, with the amount of microcrystalline cellulose adjusted accordingly; all other components were equal in each sample. The microcrystalline cellulose is a well known and widely used filler material which is not used to achieve controlled release effects. This type of filler has been shown to aid in tablet formation. Accordingly, the dramatic effect on the release rates of the various compounds can only be attributed to the variation in the amount of surfactant with respect to the acrylic polymer.

TABLE 5
Release Profile of formulations from Tables 1–4

| | | 1% SLS, | 3% SLS, | 5% SLS, |
|---|---|---|---|---|
| Time (hr) | 0% SLS, H/P = 0 | H/P = 0.02 | H/P = 0.06 | H/P = 0.1 |
| 0.5 | 33 | 28 | 24 | 17 |
| 1 | 47 | 40 | 34 | 24 |
| 2 | 66 | 57 | 49 | 32 |
| 3 | 79 | 69 | 60 | 38 |
| 4 | 88 | 78 | 68 | 43 |
| 5 | 94 | 83 | 73 | 47 |
| 6 | 98 | 87 | 77 | 51 |
| 8 | 102 | 91 | 82 | 57 |
| 10 | 103 | 93 | 86 | 61 |
| 12 | 104 | 95 | 88 | 65 |

Referring again to the FIGURE, the differences in the rate of release between the various compounds become readily apparent as early as one half hour where the control sample released approximately 33% of its active ingredient compared to approximately 28%, 24%, and 17% respectively for the 1% (0.02 S/P), 3% (0.06 S/P), and 5% (0.1 S/P) samples. After three hours, the control releases about 80% of its active ingredient. The SLS containing samples exhibit a nearly congruent path compared to the control, shifted due to increased controlled release effects. The 1% (0.02 S/P) and 3% (0.06 S/P) sample released approximately 70 and 60% of their active ingredients, respectively, after three hours. The change in the rate of release is even more dramatic in the 5% (0.1 S/P) sample, and released less than 40% of its active ingredient in the same period. It is clear that manipulation of release rates is possible through controlling the ratio of SLS to polymer in the composition.

What is claimed is:

1. A controlled release composition comprising:
a controlled release matrix comprising:
a pharmaceutically acceptable acrylic polymer; and
a surfactant in an amount selected, relative to the amount of said acrylic polymer, for and corresponding to a pre-determined release rate for said active ingredient.

2. The composition of claim 1 wherein the ratio of said surfactant to said acrylic polymer is about 0.005-0.5 by weight.

3. The composition of claim 1 wherein the ratio of said surfactant to said acrylic polymer is about 0.02-0.2 by weight.

4. The composition of claim 1 further comprising a therapeutic amount of an active ingredient.

5. The composition of claim 4 wherein said active ingredient is substantially evenly dispersed in said controlled release matrix.

6. The composition of claim 4 wherein said active ingredient is selected from the group consisting of oxycodone, oxymorphone, morphine, hydromorphone, levorphanol, codeine, hydrocodone, oxycodone, nalorphone, naloxone, naltrexone, buprenorphine, butorphanol, nalbuphine and their pharmaceutically active salts.

7. The composition of claim 1 wherein said active ingredient is oxycodone or its pharmaceutically active salts.

8. The composition of claim 1 wherein said controlled release matrix is a non-therapeutic excipient.

9. The composition of claim 1 wherein said acrylic polymer is an ammonio methacrylate based polymer.

10. The composition of claim 1 wherein said surfactant is an anionic surfactant.

11. The composition of claim 1 wherein said surfactant is sodium lauryl sulfate.

12. The composition of claim 1 wherein said surfactant is docusate sodium.

13. The composition of claim 1 wherein said surfactant is present at about 1-5% by weight of said composition.

14. A controlled release composition comprising:
a therapeutic amount of an active ingredient; and
a controlled release matrix comprising:
a pharmaceutically acceptable acrylic polymer; and
a surfactant in an amount selected for and corresponding to a pre-determined release rate of said active ingredient such that the ratio of said surfactant to said acrylic polymer is about 0.005-0.5 by weight.

15. The composition of claim 13 wherein said acrylic polymer is an ammonio methacrylate based polymer.

16. The composition of claim 12 wherein said surfactant is sodium lauryl sulfate.

17. The composition of claim 12 wherein said ratio of surfactant to acrylic polymer is about 0.02-0.2.

18. The composition of claim 12 wherein said active ingredient is selected from the group consisiting of oxycodone, oxymorphone, morphine, hydromorphone, levorphanol, codeine, hydrocodone, nalorphone, naloxone, naltreone, buprenorphine, butorphanol, nalbuphine, and their pharmaceutically active salts.

19. The composition of claim 12 wherein said active ingredient is oxycodone or its pharmaceutically active salts.

20. A controlled release therapeutic composition comprising:
a therapeutic amount of oxycodone or its pharmaceutically active salts; and
a controlled release matrix comprising:
a pharmaceutically acceptable ammonio methacrylate based polymer; and
sodium lauryl sulfate in an amount selected for and corresponding to a pre-determined release rate for the oxycodone or its pharmaceutically active salts, such that the ratio of said sodium lauryl sulfate to said ammonio methacrylate based polymer is about 0.005-0.5 by weight.

21. The composition of claim 18 wherein said ratio of said sodium lauryl sulfate to said ammonio methacrylate based polymer is about 0.02-0.2 by weight.
